(19) United States
(12) Patent Application Publication
(10) Pub. No.: US 2024/0335321 A1
(43) Pub. Date: Oct. 10, 2024

(54) INSTRUMENT FOR OPENING A CAPSULAR BAG IN AN EYE, AND TREATMENT DEVICE INCLUDING THE INSTRUMENT

(71) Applicant: Carl Zeiss Meditec AG, Jena (DE)

(72) Inventors: Matthias Hillenbrand, Jena (DE); Ernar Amanov, Heidelberg (DE); Philipp Brenner, Karlsruhe (DE)

(21) Appl. No.: 18/624,759

(22) Filed: Apr. 2, 2024

(30) Foreign Application Priority Data
Apr. 6, 2023 (DE) 10 2023 108 809.2

(51) Int. Cl. A61F 9/007

(52) U.S. Cl. CPC A61F 9/00754; A61F 9/00763

(57) ABSTRACT

An instrument is for opening a capsular bag in an eye, including a cannula configured to be inserted into the eye in an insertion direction of the cannula. The cannula has a cannula longitudinal end arranged in the insertion direction and a cannula opening arranged in a cannula portion extending from the cannula longitudinal end. A rotor is arranged within the cannula and is rotatable about a rotational axis. The rotor longitudinal end is arranged in the direction of the axis of rotation and has a deployed state wherein the rotor extends through the cannula opening and protrudes from the cannula. A gripping device is arranged outside of the cannula in the deployed state and is configured to grip a part of the capsular bag, with the rotor being configured to wind up the part of the capsular bag by virtue of the rotor rotating.

### CROSS-REFERENCE TO RELATED APPLICATIONS

This application claims priority of German patent application no. 10 2023 108 809.2, filed Apr. 6, 2023, the entire content of which is incorporated herein by reference.

### TECHNICAL FIELD

The disclosure relates to an instrument for opening a capsular bag in an eye and to a treatment device having the instrument.

### BACKGROUND

In a cataract treatment, the natural lens of an eye is replaced with an artificial intraocular lens. This requires performing a capsulorhexis in order to introduce an opening in the capsular bag in the eye, through which the natural lens is removed and the intraocular lens is subsequently introduced into the capsular bag. During capsulorhexis, puncturing or incising creates an initial opening in the capsular bag in the eye, and the initial opening is increased in size until the opening has a diameter of approx. 5 mm by repeatedly rupturing the capsular bag via a tool, for example a bent needle (cystotome) or a pair of forceps. The capsular bag must be gripped multiple times by the tool in the process, and a tear may be created in the capsular bag by way of a movement of the tool away from the natural lens. Little available depth in the anterior chamber means that gripping is a challenge because the tool must be lifted to this end, and the latter comes quite close to a sensitive structure such as endothelial cells in the process. Additionally, depth perception is a challenge for a surgeon, and so it can be difficult to safely lift the tool off, and reposition the tool on, the capsular bag. Further, a successful outcome of the safe repositioning must include the tool penetrating far enough into the capsular bag in order to enable a reliable tear. Moreover, the trajectory of the tool deviates from the trajectory of the point at which the tear arises, as a result of which a physician performing the cataract treatment must perform a complicated movement with the tool. Overall, it is complicated to perform the capsulorhexis using the bent needle or the forceps.

### SUMMARY

It is thus an object of the disclosure to develop an instrument and a treatment device with which a capsulorhexis is easily performable.

The instrument according to the disclosure for opening a capsular bag in an eye includes a cannula and a rotor. The cannula is configured to be introduced into the eye in an insertion direction of the cannula. The cannula includes a cannula longitudinal end arranged in the insertion direction and a cannula opening arranged in a cannula portion extending from the cannula longitudinal end. The rotor is arranged within the cannula so as to be rotatable about an axis of rotation and includes a rotor longitudinal end arranged in the direction of the axis of rotation. Moreover, the rotor has a deployed state in which the rotor extends through the cannula opening and consequently protrudes from the cannula and in which the rotor longitudinal end is arranged outside of the cannula. The rotor includes a gripping device arranged outside of the cannula in the deployed state and configured to grip a part of the capsular bag, with the rotor being configured to wind up the part of the capsular bag by virtue of the rotor rotating.

As a result of providing the rotor which rotates during the capsulorhexis, it is for example conceivable to guide the instrument in such a way during the capsulorhexis that the instrument is kept at a constant distance from a point at which the capsular bag tears. Such a trajectory of the instrument is less complex than in the case of a conventional tool, where the tool must be moved away from the capsular bag. Moreover, in comparison with a conventional tool, an increased length of the part of the capsular bag can be torn every time the part of the capsular bag is gripped. As a result, the capsular bag needs to be gripped less frequently. Consequently, the instrument according to the disclosure makes it easier to perform the capsulorhexis than the conventional tool.

The gripping device preferably includes a tip, a pair of forceps and/or a hook. It is also possible to introduce an initial opening into the capsular bag using these gripping devices. It is also possible to provide a plurality of these hooks, which are arranged on the rotor like in the case of a hook-and-loop fastener.

It is particularly preferable for the rotor to include a rotor interior arranged within the rotor. In this context, it is particularly preferable for the gripping device to be formed by the rotor interior, a rotor gripping opening arranged outside of the cannula in the deployed state and via which the rotor interior is accessible from outside of the rotor, and a suction device configured to suck in a part of the capsular bag. By virtue of the rotor gripping opening being blocked by the part of the capsular bag, it is possible to create a negative pressure in the rotor interior using the suction device, and so the part of the capsular bag adheres to the rotor and thus can be gripped by the rotor. For example, the suction device might be arranged in a handpiece of the instrument, with a physician performing a cataract treatment being able to grip the handpiece in their hand.

The instrument preferably includes an intermediate space arranged between the cannula and the rotor and accessible via the cannula opening. In this context, it is conceivable for the instrument to be configured to irrigate or aspirate the eye via the intermediate space and the cannula opening. By preference, the rotor includes a rotor opening arranged in a rotor portion extending from the rotor longitudinal end and via which the rotor interior is accessible, with the instrument being configured to irrigate or aspirate the eye via the rotor interior and the rotor opening. Particularly preferably, the instrument includes the intermediate space, the rotor includes the rotor opening, and the instrument is configured to irrigate the eye via the intermediate space and the cannula opening and aspirate the eye via the rotor interior and the rotor opening, or to aspirate the eye via the intermediate space and the cannula opening and irrigate the eye via the rotor interior and the rotor opening. This last-mentioned alternative is useful if a surgeon wishes to detach the part of the sucked-on capsular bag from the instrument again. Irrigation is understood to mean the supply of a fluid, in particular a liquid, to the eye, and aspiration is understood to mean the sucking of the fluid, in particular the liquid, out of the eye. Aspiration can be used to suck in the part of the capsular bag, whereas the irrigation can be used to resupply a fluid inadvertently sucked out during aspiration in order to keep a pressure variation in the eye interior low.

In a first variant of the disclosure, it is preferable for the rotor to be arranged in the cannula so as to be longitudinally displaceable and include a retracted state in which the rotor longitudinal end is arranged fully within the cannula and at a distance from the cannula opening. In a second variant of the disclosure, it is preferable for the instrument to include a contact ring arranged at a distance from the cannula longitudinal end in the insertion direction, with the rotor and the contact ring being arranged so as to be longitudinally displaceable relative to one another, with the rotor extending through the contact ring and the rotor longitudinal end protruding from the contact ring in the insertion direction in the deployed state and with the rotor having a retracted state in which the rotor longitudinal end is arranged at a position proximally of the distal end of the contact ring. It is conceivable in both variants to grip the capsular bag using the gripping device when the rotor is in the deployed state. Subsequently, the rotor can be longitudinally displaced into the retracted state and the capsular bag can be contacted with the cannula longitudinal end according to the first variant and with the distal end of the contact ring according to the second variant. Subsequently, the rotor can be rotated. As a result, it is possible to maintain a virtually constant distance between the rotor and the point at which the capsular bag tears. This allows the course over which the capsular bag tears to be controlled particularly accurately. It is particularly preferable for the contact ring to be mounted so as to be longitudinally displaceable relative to the cannula in the insertion direction, whereby the rotor and the contact ring are arranged so as to be longitudinally displaceable relative to one another. As a result, it is possible to switch the rotor particularly easily between the deployed state and the retracted state.

The instrument preferably includes an articulation which is arranged in a further rotor portion extending from the rotor longitudinal end and which divides the instrument into a first partial arm and a second partial arm arranged next to one another in the insertion direction, with the first partial arm and the second partial arm being pivotable relative to one another via the articulation. As a result, it is possible to simultaneously rotate the rotor and pivot the first partial arm relative to the second partial arm. This makes it easier to wind up the part of the capsular bag, and less tension arises within the capsular bag in the process. It is particularly preferable for the diameter of the rotor to become longer or larger with increasing distance from the articulation. This gives consideration to the fact that an increased length of the capsular bag is wound up at a greater distance from the articulation, whereby particularly little tension arises in the capsular bag.

The treatment device according to the disclosure includes the instrument according to the disclosure or a preferred embodiment thereof.

The instrument might be configured to be guided by a physician's hand. Alternatively, it is conceivable for the treatment device to include a surgical robot which is configured to guide the instrument.

The treatment device is preferably configured to control the rotation of the rotor using the length traveled by the instrument in the eye and/or using the torn length of the part of the capsular bag. For example, the length traveled can be determined on the basis of the movement performed by the surgical robot. In an alternative or in addition, it is conceivable that the treatment device includes a surgical microscope including an image processing unit configured to determine the length traveled by the instrument. In another example, the length traveled can be determined using an accelerometer by integrating the measured values twice. Another option consists of providing a potentiometer between the rotor and the cannula. It is conceivable that the treatment device includes a surgical microscope including an image processing unit configured to determine the torn length of the capsular bag. In another example, the torn length can also be determined via coherence tomography. In particular, the rotation of the rotor can be controlled such that the wound-up length of the part of the capsular bag corresponds to the length traveled by the instrument in the eye or to the torn length of the part of the capsular bag. What this achieves is that the distance between the instrument and the point at which the capsular bag tears is kept constant. This is advantageous because the direction of the further course of the tear can be controlled well as a result. This facilitates performing the capsulorhexis.

Alternatively, the treatment device is configured to control the rotation of the rotor such that the distance of the instrument from the position at which the capsular bag tears is kept constant. To this end, the treatment device may include a surgical microscope including an image processing unit configured to determine the point at which the capsular bag tears, the position of the instrument, and the distance between the point and the position.

By preference, the treatment device is configured during the closed-loop control of the rotation of the rotor to give consideration to the fact that, on account of wound-up layers of the capsular bag as the rotation of the rotor progresses, an increased length of the part of the capsular bag is wound up during each rotation of the rotor. For example, the treatment device may include a computing unit in which an average thickness of a capsular bag is stored and which is used to this end.

### BRIEF DESCRIPTION OF DRAWINGS

The invention will now be described with reference to the drawings wherein:

FIG. 1 shows a perspective view of a first embodiment of the instrument according to the disclosure at a first time;

FIG. 2 shows a perspective view of the first embodiment at a second time;

FIG. 3 shows a perspective view of the first embodiment at a third time;

FIG. 4 shows a side view of a second embodiment of the instrument according to the disclosure;

FIG. 5 shows a perspective view of a third embodiment of the instrument according to the disclosure at a first time;

FIG. 6 shows a perspective view of the third embodiment at a second time;

FIG. 7 shows a side view of a fourth embodiment of the instrument according to the disclosure;

FIG. 8 shows a plan view of a fifth embodiment of the instrument according to the disclosure;

FIG. 9 shows a plan view of a sixth embodiment of the instrument according to the disclosure;

FIG. 10 shows a perspective view of a seventh embodiment of the instrument according to the disclosure;

FIG. 11 shows a perspective view of an eighth embodiment of the instrument according to the disclosure at three different times, with time progressing from left to right; and,

FIG. 12 shows a section through a treatment device.

### DETAILED DESCRIPTION

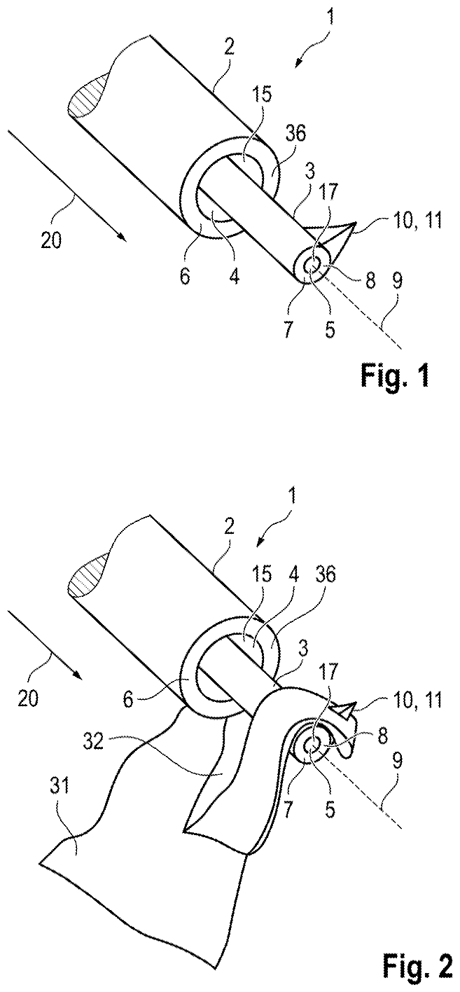

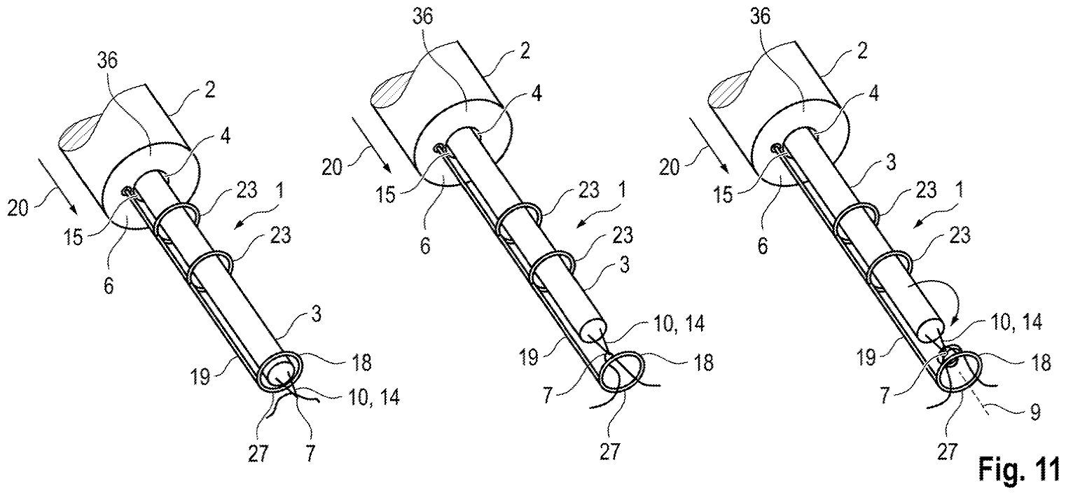

As is apparent from FIGS. 1 to 12, an instrument 1 for opening a capsular bag 31 in an eye 34 includes a cannula 2 and a rotor 3. The cannula 2 is configured to be introduced into the eye 34 in an insertion direction 20 of the cannula 2. Moreover, the cannula 2 includes a cannula longitudinal end 6 arranged in the insertion direction 20 and a cannula opening 15 (FIGS. 1 to 3, 5, 6, 10 and 11) arranged in a cannula portion extending from the cannula longitudinal end 6. The rotor 3 is arranged within the cannula 2 so as to be rotatable about an axis of rotation 9 (compare FIGS. 1 to 12). The rotor 3 includes a rotor longitudinal end 7 arranged in the direction of the axis of rotation 9 and has a deployed state in which the rotor 3 extends through the cannula opening 15 and consequently protrudes from the cannula 2 and in which the rotor longitudinal end 7 is arranged outside of the cannula 2. The deployed state is depicted in FIGS. 1 to 10 and at the first time in FIG. 11. Moreover, the rotor 3 includes a gripping device 10 (see FIGS. 1 to 11) arranged outside of the cannula 2 in the deployed state and configured to grip a part of the capsular bag 31, with the rotor 3 being configured to wind up the part of the capsular bag 31 by virtue of the rotor 3 rotating.

The rotor 3 can be configured to rotate less than one revolution, to rotate more than one revolution or to rotate through multiple revolutions.

For example, the cannula portion can have an extent of 7 to 8 mm in the insertion direction 20 in order to achieve a capsulorhexis with a diameter of 5 mm.

For example, the rotor 3 can have a Young's modulus ranging from 40*10⁹ Pa to 200*10⁹ Pa. As a result, it is possible that the rotor 3 extends through a curved region of the cannula 2 and is also able to rotate within the curved region. This Young's modulus also allows the rotor 3 to be displaced longitudinally in the cannula 2 despite the curved region.

It is conceivable that the cannula 2 is beveled in the region of the cannula longitudinal end 6 by virtue of the cannula 2 having a cannula end face 36 which directly adjoins the cannula opening 15 and whose normal makes an angle that differs from 0° with the axis of rotation 9. As a result, the cannula 2 can be inserted particularly easily into the eye 34. In particular, the angle is in a range from 10° to 80°.

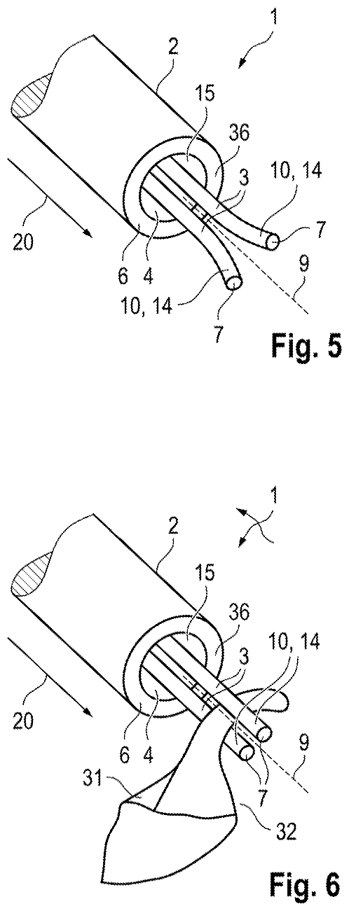

FIGS. 1, 2 and 4 show that the gripping device 10 may include a tip 11. The tip 11 may become narrower as the distance from the axis of rotation 9 increases. FIGS. 5, 6 and 11 show that the gripping device 10 may include a pair of forceps 14. The forceps 14 may include two arms which have a spaced-apart state, in which the two arms are spaced apart from one another (see FIG. 5), and a proximity state, in which the two arms are in proximity and in which the part of the capsular bag 31 can be clamped by the two arms (see FIGS. 6 and 11). Clamping can be implemented by pulling the arms into the cannula.

The rotor 3 may include a rotor interior 5 arranged within the rotor 3; compare FIGS. 1 to 3 and 10. FIG. 10 shows that the gripping device 10 can be formed by the rotor interior 5, a rotor gripping opening 16 arranged outside of the cannula 2 in the deployed state and via which the rotor interior 5 is accessible from outside of the rotor 3, and a suction device configured to suck in the part of the capsular bag 31. The rotor interior 5 may be introduced in a circumferential area of the rotor 3—compare FIG. 10—such that the rotor interior 5 is accessible from outside of the rotor 3 in a direction perpendicular to the axis of rotation 9.

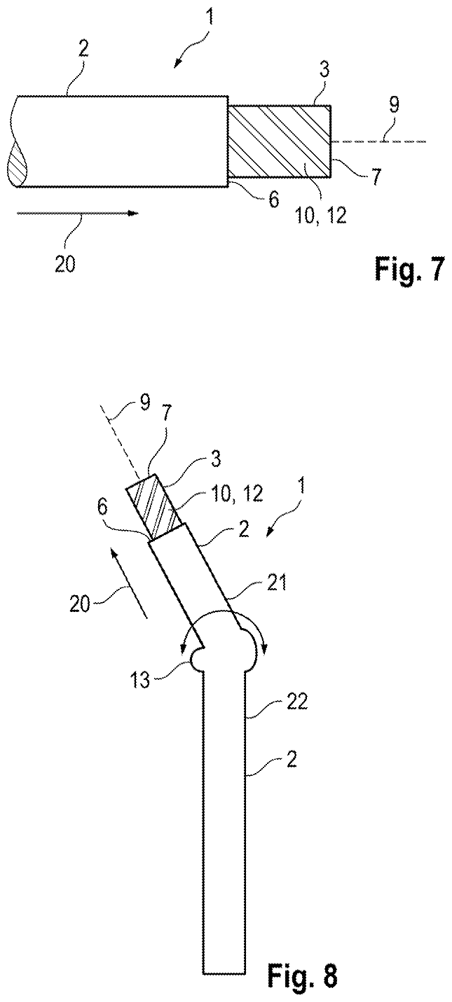

The gripping device 10 may include one hook or a plurality of hooks. In the case of the plurality of hooks, it is conceivable that the hooks are arranged on the rotor 3 like in the case of a hook-and-loop fastener and consequently form a structured surface 12 of the rotor 3; compare FIGS. 7 to 9.

FIGS. 1 to 3, 5, 6, 10 and 11 show that the instrument 1 may include an intermediate space 4 arranged between the cannula 2 and the rotor 3 and accessible via the cannula opening 15. It is conceivable for the instrument 1 to be configured to irrigate or aspirate the eye 34 via the intermediate space 4 and the cannula opening 15. The rotor 3 may include a rotor opening 17 arranged in a rotor portion extending from the rotor longitudinal end 7 and via which the rotor interior 5 is accessible. It is conceivable for the instrument 1 to be configured to irrigate or aspirate the eye 34 via the rotor interior 5 and the rotor opening 17. For example, the rotor portion may have an extent of 5 mm or 3 mm in the insertion direction 20. In particular, the rotor opening 17 may be introduced in a rotor end face 8 of the rotor 3, with the rotor end face 8 forming the rotor longitudinal end 7; see FIGS. 1 to 3. In particular, the instrument 1 may be configured to irrigate the eye 34 via the intermediate space 4 and the cannula opening 15 and aspirate the eye via the rotor interior 5 and the rotor opening 17, or to aspirate the eye 34 via the intermediate space 4 and the cannula opening 15 and irrigate the eye via the rotor interior 5 and the rotor opening 17.

It is conceivable that the rotor 3 in the deployed state protrudes from the cannula 2 in the insertion direction 20; compare FIGS. 1 to 3 and 5 to 11. In the process, the axis of rotation 9 may be arranged parallel to the insertion direction 20. Alternatively, it is conceivable that the rotor 3 in the deployed state protrudes from the cannula 2 across the insertion direction 20; compare FIG. 4. FIG. 4 shows that the cannula 2 may have a curved embodiment, in particular in order to bring about a deflection of the rotor 3 such that there is a first region, in which the axis of rotation 9 is oriented parallel to the insertion direction 20, and a second region, in which the axis of rotation 9 is arranged across, in particular perpendicular to, the insertion direction 20.

As is evident from FIG. 11, the instrument 1 may include a contact ring 18 which is arranged at a distance from the cannula longitudinal end 6 in the insertion direction 20. The rotor 3 and the contact ring 18 can be arranged so as to be longitudinally displaceable relative to one another, with the rotor 3 extending through the contact ring 18 and the rotor longitudinal end 7 protruding from the contact ring 18 in the insertion direction 20 in the deployed state and with the rotor 3 having a retracted state in which the rotor longitudinal end 7 is arranged at a position proximally of the distal end of the contact ring 18. The contact ring 18 can be mounted so as to be longitudinally displaceable relative to the cannula 2 in the insertion direction 20. The instrument 1 may include a rod 19 which is fastened to the cannula 2, in particular fastened so as to be longitudinally displaceable, and to which the contact ring 18 is fastened. Moreover, it is conceivable for the instrument 1 to include two rods or more than two of the rods 19. It is moreover conceivable that the instrument 1 includes a guide ring 23 through which the rotor 3 extends in the deployed state and which is arranged at a distance from and proximally to the contact ring 18 and arranged distally from the cannula longitudinal end 6. It is also conceivable that the instrument 1 includes a plurality of the guide rings 23, which are arranged at a distance from one another in each case. It is conceivable that the contact ring 18 has an angled bevel in the region of the distal end 27 of the contact ring 18 by virtue of the contact ring 18 having an end face which directly adjoins the contact ring opening and whose normal makes an angle that differs from 0° with the axis of rotation 9. As a result, the contact ring 18 can be inserted particularly easily into the eye 34. In particular, the angle is in a range from 10° to 80°.

As an alternative to providing the contact ring 18, the rotor 3 can be arranged in the cannula 2 so as to be longitudinally displaceable and include a retracted state in which the rotor longitudinal end 7 is arranged fully within the cannula 2 and at a distance from the cannula opening 15. However, the embodiment with the contact ring 18 is advantageous in that the part of the capsular bag 31 can reach into a free region between the contact ring 18 and the cannula longitudinal end 6 when being wound onto the rotor 3, and hence it is possible to prevent the cannula 2 becoming blocked by the part of the capsular bag 31.

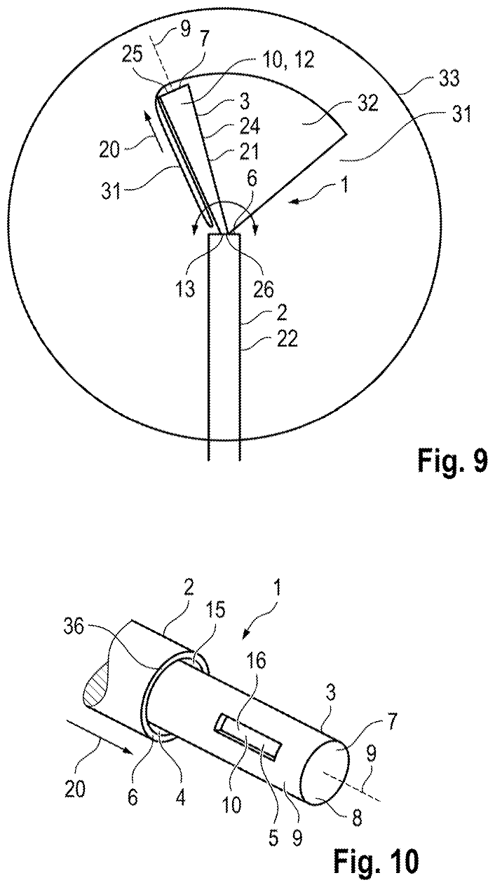

FIGS. 8 and 9 show that the instrument 1 may include an articulation 13 which is arranged in a further rotor portion extending from the rotor longitudinal end 7 and which divides the instrument 1 into a first partial arm 21 and a second partial arm 22 arranged next to one another in the insertion direction 20, with the first partial arm 21 and the second partial arm 22 being pivotable relative to one another via the articulation 13. For example, the instrument 1 may include a Bowden cable or two rods arranged in the cannula 2 for the purpose of driving the articulation 13. The first partial arm 21 and the second partial arm 22 can be pivoted relative to one another by pulling the Bowden cable or pressing one of the rods. Alternatively, it is conceivable that the articulation 13 has two separated pressure chambers to which a pressure or negative pressure can be applied independently of the other, and which therefore change their volume and thereby drive the articulation 13. The first arm 21 may include the gripping device 10. For example, the articulation 13 might be formed by the cannula 2 (see FIG. 8) and/or by the rotor 3 (see FIG. 9). For example, in the deployed state, the further rotor portion may have an extent in the insertion direction 20 that is longer than the extent of the rotor portion in the insertion direction 20. FIG. 9 shows that the diameter of the rotor 3 can become larger with increasing distance from the articulation 13 in a region arranged outside of the cannula 2 in the deployed state. For example, the rotor 3 may have the shape of a conical frustum 24 to this end, with the base 25 of the conical frustum 24 forming the rotor longitudinal end 7. The top surface 26 of the conical frustum 24 is arranged proximally to the base 25 and, in particular, may be adjacent to the articulation 13.

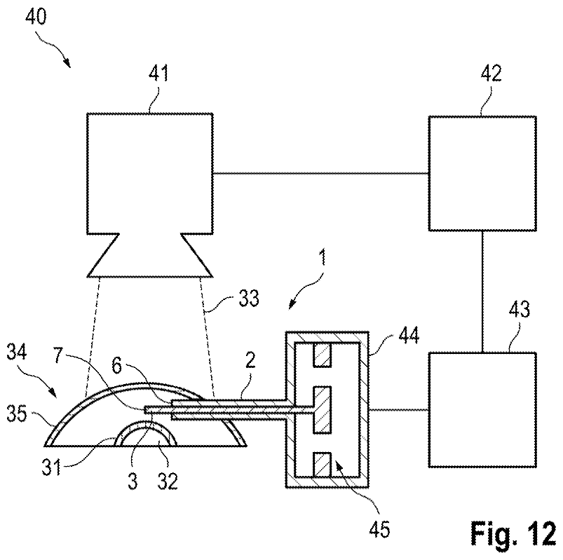

FIG. 12 shows a treatment device 40 having the instrument 1. The eye 34 with a cornea 35, the capsular bag 31 and a lens 32 is shown, with the cannula longitudinal end 6 and the rotor longitudinal end 7 of the instrument 1 being arranged in the eye 34. The instrument 1 may include a motor 45 configured to rotate the rotor 3. Moreover, the instrument 1 may include a handpiece 44 which can be gripped by the hand of a physician performing a cataract treatment. For example, the motor 45 can be arranged in the handpiece 44.

The treatment device 40 may include a surgical microscope 41 with a field of view 33 (see also FIG. 9). Moreover, the treatment device 40 may include an image processing unit 42 which is coupled to the surgical microscope 41 and configured to analyze images recorded by the surgical microscope 41.

The treatment device 40 may be configured to control the rotation of the rotor 3 using the length traveled by the instrument 1 in the eye 34 and/or using the torn length of the capsular bag 31. To this end, the treatment device 40 may include a closed-loop control unit 43 which is coupled to the motor 45 and configured to control the angular speed of the motor 45. The length traveled by the instrument 1 and/or the torn length of the capsular bag 31 can be determined using the surgical microscope 41 and the image processing unit 42, with the closed-loop control unit 43 being coupled to the image processing unit 42. However, it is also conceivable that the treatment device includes a surgical robot configured to guide the instrument 1 and that the length traveled by the instrument 1 is determined on the basis of the coordinates traversed by the surgical robot. In particular, the rotation of the rotor can be controlled such that the wound-up length of the part of the capsular bag 31 corresponds to the length traveled by the instrument in the eye or to the torn length of the capsular bag 31.

Alternatively, the treatment device 40 can be configured to control the rotation of the rotor 3 such that the distance of the instrument 1 from the position at which the capsular bag 31 tears is kept constant. To this end as well, the treatment device 40 may include a closed-loop control unit 43 which is coupled to the motor 45 and configured to control the angular speed of the motor 45. The image processing unit 42 may be coupled to the closed-loop control unit 43 and may be configured to determine the position of the instrument 1, the position at which the capsular bag 31 tears and the distance between these two positions.

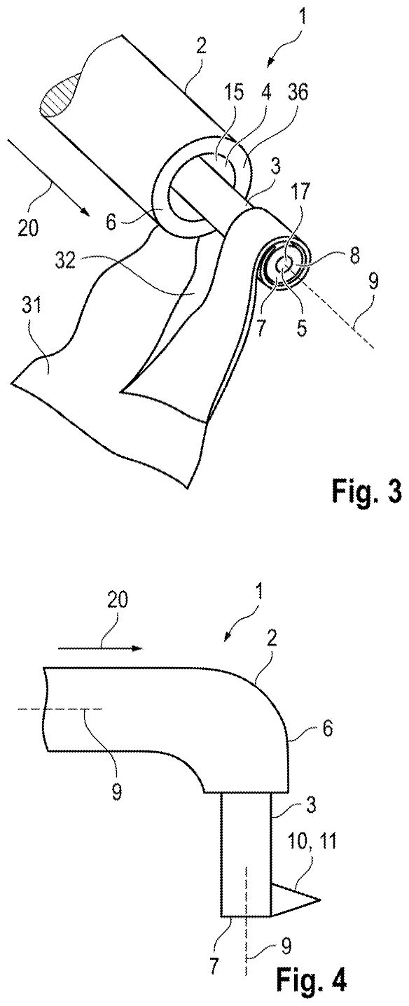

Moreover, it is conceivable that the treatment device 40 is configured during the closed-loop control of the rotation of the rotor 3 to give consideration to the fact that, on account of wound-up layers of the capsular bag 31 as the rotation of the rotor 3 progresses, an increased length of the capsular bag 31 is wound up during each rotation; compare for example FIG. 3, in which one layer of the part of the capsular bag 31 is arranged on another layer of the part of the capsular bag 31.

It is understood that the foregoing description is that of the preferred embodiments of the invention and that various changes and modifications may be made thereto without departing from the spirit and scope of the invention as defined in the appended claims.

### List of Reference Signs

1 Instrument
2 Cannula
3 Rotor
4 Intermediate space
5 Rotor interior
6 Cannula longitudinal end
7 Rotor longitudinal end
8 Rotor end face
9 Axis of rotation
10 Gripping device
11 Tip
12 Surface structuring
13 Articulation
14 Forceps
15 Cannula opening
16 Rotor gripping opening
17 Rotor opening
18 Contact ring
19 Rod
20 Insertion direction
21 First partial arm
22 Second partial arm
23 Guide ring
24 Conical frustum
25 Base
26 Top surface
27 Distal end of the contact ring
31 Capsular bag
32 Lens
33 Field of view of the surgical microscope
34 Eye
35 Cornea
36 Cannula end face
40 Treatment device
41 Surgical microscope
42 Image processing unit
43 Closed-loop control unit
44 Handpiece
45 Motor

1. An instrument for opening a capsular bag in an eye, the instrument comprising:
a cannula configured to be inserted into the eye in an insertion direction of the cannula;
said cannula having a cannula longitudinal end arranged in said insertion direction;
said cannula further having a cannula opening arranged in a cannula portion extending from said cannula longitudinal end;
a rotor defining a rotational axis and being arranged within said cannula so as to be rotatable about said rotational axis;
said rotor having a rotor longitudinal end arranged in the direction of said rotational axis;
said rotor defining a deployed state wherein said rotor extends through said cannula opening so as to protrude from said cannula and wherein said rotor longitudinal end is arranged outside of said cannula;
a gripper arranged outside of said cannula in said deployed state and being configured to grip a part of the capsular bag; and,
said rotor being configured to wind up part of said capsular bag via rotation of said rotor.

2. The instrument of claim 1, wherein said rotor defines a rotor interior arranged within said rotor.

3. The instrument of claim 2, wherein said gripper is formed by said rotor interior, a rotor gripping opening arranged outside of said cannula in said deployed state and via which said rotor interior is accessible from outside of said rotor, and a suction device configured to draw a fluid out of said rotor interior.

4. The instrument of claim 2, wherein:
said cannula and said rotor conjointly define an intermediate space therebetween accessible via said cannula opening;
said rotor includes a rotor opening arranged in a rotor portion extending from said rotor longitudinal end; and,
said instrument being configured to irrigate the eye via said intermediate space and said cannula opening and to aspirate the eye via one of the following:
i) aspiration via said rotor interior and said rotor opening; or,
ii) aspirate the eye via said intermediate space and said cannula opening and irrigate the eye via said rotor interior and said rotor opening.

5. The instrument of claim 1, wherein one of the following applies:
i) said rotor is arranged in said cannula so as to be longitudinally displaceable and includes a retracted state wherein said rotor longitudinal end is arranged fully within said cannula and at a distance from said cannula opening; or,
ii) said instrument includes a contact ring arranged at a distance from said cannula longitudinal end in the insertion direction, with said rotor and said contact ring being arranged so as to be longitudinally displaceable relative to one another, with said rotor extending through said contact ring and said rotor longitudinal end protruding from said contact ring in said insertion direction in said deployed state and with said rotor having a retracted state wherein said rotor longitudinal end is arranged at a position proximally of a distal end of said contact ring.

6. The instrument of claim 1, wherein the instrument further comprises an articulation arranged in a further rotor portion extending from said rotor longitudinal end and dividing said instrument into a first partial arm and a second partial arm arranged next to one another in said insertion direction; and, said first partial arm and said second partial arm being pivotable relative to one another via said articulation.

7. The instrument of claim 6, wherein said rotor has a diameter becoming longer with increasing distance from said articulation.

8. A treatment device comprising:
an instrument;
said instrument including:
a cannula configured to be inserted into the eye in an insertion direction of the cannula;
said cannula having a cannula longitudinal end arranged in said insertion direction;
said cannula further having a cannula opening arranged in a cannula portion extending from said cannula longitudinal end;
a rotor defining a rotational axis and being arranged within said cannula so as to be rotatable about said rotational axis;
said rotor having a rotor longitudinal end arranged in the direction of said rotational axis;
said rotor defining a deployed state wherein said rotor extends through said cannula opening so as to protrude from said cannular and wherein said rotor longitudinal end is arranged outside of said cannula;
a gripper arranged outside of said cannula in said deployed state and being configured to grip a part of the capsular bag; and,
said rotor being configured to wind up part of said capsular bag via rotation of said rotor.

9. The treatment device of claim 8, wherein said treatment device is configured to control the rotation of said rotor using the length traveled by said instrument in the eye and/or using the torn length of the part of the capsular bag, or wherein said treatment device is configured to control the rotation of said rotor such that the distance of said instrument from the position at which the capsular bag tears is kept constant.

10. The treatment device of claim 9, wherein said treatment device is configured during closed-loop control of the rotation of the rotor to give consideration to the fact that, on account of wound-up layers of the capsular bag as the rotation of the rotor progresses, an increased length of a part of the capsular bag is wound up during each rotation.
